(19) United States
(12) Patent Application Publication
(10) Pub. No.: US 2014/0347630 A1
(43) Pub. Date: Nov. 27, 2014

(54) Fast measurement of ocular axial length

(71) Applicants: Alessandro Foggi, San Giovanni Valdarno (AR) (IT); Luca Pezzati, Firenze (FI) (IT)

(72) Inventors: Alessandro Foggi, San Giovanni Valdarno (AR) (IT); Luca Pezzati, Firenze (FI) (IT)

(73) Assignee: VISIA IMAGING S.R.L., San Giovanni Valdarno (AR) (IT)

(21) Appl. No.: 14/373,738

(22) PCT Filed: Jan. 24, 2013
(86) PCT No.: PCT/IB2013/050621

§ 371 Date: Jul. 22, 2014

(30) Foreign Application Priority Data
Jan. 24, 2012 (IT) PI2012A000009

(51) Int. Cl. A61B 3/10; A61B 3/00; A61B 3/14

(52) U.S. Cl. CPC A61B 3/1005; A61B 3/14; A61B 3/0025; A61B 3/0075
USPC 351/206; 351/205; 351/246

(57) ABSTRACT

The present invention concerns a Michelson-type interferometer (1) for measuring the intraocular axial length (AL) comprising: an arrangement (7) able to move apart and come near with respect to an emitting light source (5), said arrangement (7) comprising at least a first (8) and second (9) at least partially reflecting surface arranged at a pre-determined mutual distance (d); a motorized driving system (500) to command the movement of the reflecting arrangement (7); wherein said motorized driving system (500) is controlled in such a way that the scanning is completed in a single translation stroke in a direction starting from an initial position in which the first surface (8) generates the first interference peak at the beginning of the translation and with such a fixed distance (d) between the plates that afterwards during said translation the second plate (9) generates the second interference peak.

### TECHNICAL FIELD

The present invention refers to the technical field relative to optical measuring instruments, in particular ophthalmology instruments with which the parameters necessary for designing intraocular lenses to be installed in cataract surgeries are detected.

In particular, the invention refers to an innovative reflecting element to be used in Michelson-type interferometers.

### BACKGROUND ART

More or less complex machineries for measuring parameters for designing intraocular lenses have long been known.

Such machineries permit the calculation of the following essential parameters for designing an ocular lens:

1) Keratometry, which is the measuring of the three sizes Rf, Rs, Δk. This measure serves to evaluate the shape of the cornea through the detection of the parameters Rf and Rs (flatter meridional radius and curver meridional radius) and of the parameter Δk that represents the difference between the said two radiuses.

2) Anterior chamber central depth (ACD): this parameter measures the intraocular distance between the posterior face of the cornea and the anterior face of the crystalline lens, which is generally of the order of about 2÷4 mm.

3) Axial length (AL): this third parameter, instead, measures the intraocular axial distance between the external surface of the cornea and the retina, whose average distance is of about 25 mm.

The exact knowledge of these parameters helps designing an intraocular lens that better interprets the refractive features of each patient.

The known machineries generally integrate among them various parts, each one of which is appointed to evaluate one of the parameters listed above.

In particular, such machineries foresee a support frame on which the measuring instrument is arranged and a support on which to place the chin and opposite the patient so as to allow a comfortable positioning of the eye with respect to the aiming system.

One of the known principles on which the measuring of the axial length is based is low-coherence interferometry, and in particular the well-known Michelson interferometer is used in its configuration in optical fiber.

The Michelson interferometer in optical fiber is composed of four branches of optical fiber crossed by coherent or incoherent electromagnetic radiation (e.m.). The four branches depart from a central node called optical fibers coupler. An inlet branch is connected to a light source coupled in fiber. Such a source is a super-luminescent diode (SLED) that emits radiation around a wave length of 820 nm. The radiation emitted by the SLED is directed to the coupler, where it suffers a division into two opposed branches: about the 10% of the energy enters in the low-intensity branch, while the 90% enters in the other branch. The radiation in exit from the low-intensity branch is collimated by a lens and directed to the eye of the patient, while the radiation in exit from the other branch is collimated by a second lens and directed to the reference surface (mobile) of the interferometer. This is generally a low-reflectance element (about the 4%) constituted of a single flat plate of optical glass (or another optical material) translatable along a guide parallel to the axis of the collimated beam exiting from the fiber.

The radiation reflected by the ocular surfaces of the patient, in good alignment conditions, re-enters the fiber crossing in the inverse sense the lens of collimation of the low-intensity branch. The radiation reflected by the reference surface also re-enters the other branch crossing the second lens. The two beams of re-enter crossing the coupler reunite in this way: 90% of the energy coming from the eye reunite with the 10% of the energy coming from the reference surface. This radiation, present in the fourth branch of the interferometer, is directed to a photodiode that measures its intensity, in turn connected to an amplifier, to an analog/digital converter (ADC) and to an electronic processor.

The collimator that aims at the eye is fixed at a pre-determined distance in such a way that the two arms of the interferometer have an almost equal difference of optical path, the arm of the plate being shorter of a few millimeters.

Both the ocular surfaces and the plate reflect back certain quantities of radiation that, through the two collimators, re-enter the optical fibers and by re-combining through the central node they are directed to the photodiode. During the measuring the plate translates in such a way as to reach the position in which its optical distance from the source is equal to that of the external surface of the eye. In this condition, the interferometer has equal arms and produces interference with any type of radiation it is illuminated (coherent or incoherent). In this first position, in particular, the two waves reflected (that of the external surface of the cornea and that of the plate) overlap in constructive interference, generating a maximum of signal.

Going on with the translation, other maximums of signal are obtained in all those positions in which the optical distance between the reference surface and one of the intraocular surfaces that are the object of the measuring is identical. The temporal tracing of the signal obtained by the interferometer during the translation of the reference plate contains a series of maximums, in correspondence of all the axial positions of the intraocular surfaces to measure. In particular, there are two intense maximums in correspondence of the anterior surface of the cornea and of the surface of the retina. All the maximums are surrounded by an envelope having an oscillating shape and caused by secondary minimums and maximums of the interference, due to the partial coherence of the light emitted by the SLED. The secondary maximums, in the case in question, are about a hundred for each main maximum. By analyzing the envelope with appropriate known mathematical techniques, the position of the main peak is obtained. Last, by knowing the scanning speed, or alternatively the position of each point of the temporal tracing (for example, through an appropriate sampling, executed by using the signal of an encoder) the optical distance of the main peaks can be deduced, from which the geometric distance of the axial length (AL) sought is obtained.

A problem of the measuring with this technology resides in the fact that the distance that the reference plate has to cover to measure the axial length also of eyes bigger than the average ones is relatively long, of the order of the 45 mm. During the scanning time, in which the plate covers said distance, it is easy that, in a way unnoticed, the patient rotates or more generically moves the eye with a sudden and uncontrolled movement. This movement of the eye during the measuring causes the registration of incorrect distances between the intraocular surfaces measured.

It is to be highlighted that a simple solution to said technical problem that foresees an increase in the scanning speed is not viable since there would be problems of detection of a very-high-frequency signal. The signal that increases in frequency is the carrier of the envelope due to the alternation of clear and dark edges in the profile.

Therefore, there is actually the need of a device, in particular of a reflecting arrangement, which results configured in such a way as to reduce as much as possible, preferably to halve, the covering time currently required without increasing the scanning speed.

A solution, for example, has been proposed in US patent application no. US2005/0140981 filed on 30 Jun. 2005. This application addresses the technical problem of how to obtain an equipment for ophthalmic measurements that is versatile and that does not require the arrangement of an excessive number of components placed in front of the patient. The solution is obtained with a modular apparatus in which the various components are placed at a distance and are among them connectable and detachable. One of these components also foresees a Michelson interferometer comprising an arrangement on which two reflecting plates are arranged, one in front of the other and adjustable each time at distances different from each other. On said two plates the beam of light coming from a branch of the interferometer is sent, while a second beam of light, through two lenses, is addressed to the two optical surfaces of which the distance wants to be measured. The two reflecting plates have to be each time adjusted at a reciprocal distance (distance d2) which more or less coincides with the distance that it is expected to be measured (in the example given in paragraph [0070] an optical distance of 34 mm is indicated).

In this way, it is as if there were two interferometers in parallel and therefore it is not necessary that the single plate makes a long path to intercept the two surfaces to be measured as per the background art. The method foresees an initial adjustment of the distance between the plates coinciding with the distance that is expected to be found to then make a high-frequency oscillation of the plates in such a way as to obtain a measuring distance at the end of the oscillation.

The arrangement is therefore made to oscillate at a frequency of about 10 Hz in such a way that each plate oscillates around the surface of which the measuring wants to be made. During the oscillation every time that the optical path between the light reflected by the first plate and the light that comes from the first optical surface is the same there is an interference peak. The same thing takes place when the optical path between the reflected light coming from the second plate and the second optical surface is the same. An envelope of peaks is thus created, which allows somehow to obtain the ocular distance that wants to be measured.

It is obvious that this solutions has anyway significant technical inconveniences.

The first among all of them is that it is necessary, every time, to adjust the two plates at a relative distance that more or less coincides with the intraocular distance that is expected to be found in the measuring. This is so because the method foresees an oscillation around the surface that wants to be measured with the obvious need to have to adjust every time a new distance between the plates on the basis of the distance that is expected to be found.

It is obvious that a manual adjustment, every time, is not a comfortable operation and it affects in a significant way the precision of the measuring. The human eye in fact foresees intraocular distances that are very different from subject to subject. Although a sort of “average” of the distances can exist, in a pre-determined subject a certain intraocular distance can be very different with respect to that of another patient (for example, if the patient is an adult or a child). This means that the initial adjustment between the two plates on the basis of the distance that is expected to be measured is an operation that inevitably takes to an erroneous measuring with the risk of making an oscillation that will never intercept the optical surface. If it is expected to measure a pre-determined distance d1 and then in the patient his actual distance is very different from the one that was expected, the standardized adjustment of the plates (distance d2) can result absolutely inappropriate for that patient. In this case, the oscillation of the plates could not intercept any optical surface or even intercept optical surfaces different from the ones that want to be measured, thus obtaining an absolutely erroneous result.

Therefore, the use of an oscillation with a manual adjustment of the distances between the plates on the basis of the distance that is expected to be found affects the precision of the measuring.

Moreover, the method described in US2005/0140981 requires a high-frequency oscillation for a relatively long time, and, during this time, the patient could move the eye, generating a further error in the measuring.

### DISCLOSURE OF INVENTION

It is therefore the aim of the present invention to provide a new interferometric device of the “Michelson” type that allows to solve, at least in part, said technical inconveniences.

In particular, it is the aim of the present invention to provide a new interferometric device in which the motion required to the reflecting element or arrangement results significantly reduced (even halved) with respect to that of the background art, though resulting particularly simple from the point of view of structure and allowing at the same time not to have to increase the scanning speed beyond practical limits.

It is therefore the aim of the present invention to provide a new interferometric device, and relative method, in which the measuring obtained does not require high-frequency oscillations and manual adjustments of the distances between the plates on the basis of the distances to be measured, thus resulting very precise; the reduction of the time necessary to make it is also obtained.

In particular, it is the aim of the present invention to provide a new interferometric device, and relative method, in which there is not the risk, during the measuring, of not intercepting any optical surface; or even erroneous optical surfaces with respect to those that are expected to be measured.

These and other aims are therefore reached with the present interferometric device for measuring an axial length (AL) as per claim 1.

The interferometer that is the object of the invention, comprising an element (7) at least partially reflecting and translatable along a motion direction in such a way that it can move apart or come near with respect to an emitting light source (5). Said element (7) foresees at least a first surface (8) and at least a second surface (9), which are both at least partially reflecting. The two surfaces are arranged at a pre-determined reciprocal distance (d).

Moreover, the two surfaces (8, 9) are arranged in the arrangement (7) in such a way as to keep said reciprocal distance (d) constant at least during the translation of the arrangement (7) in the scanning phase.

In accordance with the invention, a motorized guide system (500) is foreseen to command the movement of the reflecting arrangement (7) which is controlled in such a way that the scanning is completed in a single motion of translation in a direction starting from an initial position in which the first surface (8) generates the first interference peak at the beginning of the translation and with such a fixed distance (d) between the plates so that afterwards during said translation the second plate (9) generates the second interference peak.

Unlike the background art described, therefore, there is no oscillation around the optical surfaces in order to complete the scanning but instead a single motion of translation is made starting from a starting point in which the first plate immediately generates interference. The distance between the plates is fixed and is such that afterwards, during the translation, the second interference peak is generated. Such a stroke is enough for intercepting the two optical surfaces of interest and thus generate the interference peaks that allow to obtain to the axial length (AL).

By single stroke of translation in a direction is meant not a high-frequency oscillation but a single forward motion. The fact remains that, in order to optimize the measuring, it is possible to make two or three strokes, for example (three forward ones and three return ones). The measuring is completed already at the first forward motion but the measurings obtained in the remaining strokes can be used to obtain the verification of the calculation found or an envelope that optimizes the calculation. Nevertheless, regular high-frequency oscillations are not made.

In this way, the measuring is quick and precise. There is not the risk of not intercepting any surface and it is not required to modify the distance between the plates for different measures.

During the translation of the entire element (7) to make the measuring, the posterior reflecting element (9) reaches almost immediately the position in which its distance from the source is equal to the one of the retina going in constructive interference and producing the peak that measures the reciprocal distance with respect to the peak generated by the anterior surface (8) when the cornea is reached.

This solution is as if a simultaneous use of two separate interferometers was actually allowed and of which the “output” are however combined in a unique tracing containing the envelopes relative to all the peaks generated by the reset of the difference of optical path of the first (8) and of the second (9) surface during the scanning.

The first surface (8), as soon as it intercepts the cornea, generates a first peak, while the second surface, in virtue of the distance (d) at which it is positioned with respect to the first one (8), immediately intercepts the retina, generating the second peak. It is clear that the detection of the two peaks, used in a specific formula to obtain the axial distance sought, takes place in accordance with said solution in half the time with respect to the background art described.

Such a solution is therefore structurally simple and economical and allows to reduce significantly or even to halve the scanning distance covered by the reference surface, the whole at the same scanning speed. The time necessary to operate the measuring is therefore reduced significantly, reducing to the minimum the risk of accidental movement of the eye of the patient during the measuring.

Advantageously, the first (8) and the second surface (9) can be placed parallel and/or coaxial between them.

Advantageously, there can be foreseen adjusting means for allowing a reciprocal sliding of the two surfaces (8, 9) in the arrangement (7) in such a way as to adjust their reciprocal distance (d), and fixing means to block said reciprocal distance or, alternatively, be directly fixed.

Advantageously, said reflecting arrangement (7) has an optical length comprised between the 15 mm and the 25 mm.

Advantageously, the distance between the first (8) and the second surface (9) is comprised between the 12 mm and the 19 mm.

Advantageously, the motorized system (500) is connected to an electronic processor (PC) that commands the movement between two extreme positions.

Advantageously, the following are further foreseen:
A system of acquisition/analysis of the images (300, 400) configured to acquire a plurality of images at different focal distances and select the image that results in focus;
A translation system (210) that allows to translate the interferometer (1) on the basis of the images acquired in such a way that when the image acquired results in focus the interferometer results positioned with the first surface (8) substantially at an interference distance with respect to the first surface (100) of the axial length (AL) to be detected.

Advantageously, a frame is foreseen on which said acquisition/analysis system of the images and the interferometer (1) are foreseen, said frame being reciprocable in such a way as to allow the acquisition of images at different focal distances.

It is also here described a method for measuring the intraocular axial length (AL) by means of a Michelson interferometer (1) comprising an arrangement (7) at least partially reflecting of the light and provided with at least a first (8) and with at least a second (9) surface at least partially reflecting, arranged between them at a pre-determined reciprocal distance (d), said method comprising the following operations:
Initial positioning of the reflecting arrangement (7) in such a way that the first reflecting surface (8) is found at a distance (d″) from the collimator (5) inferior to the distance (d′) of the collimator (4) from the cornea (100) of the eye;
Sending of the light beam through the two branches (4′, 5′) terminating in the two collimators (4, 5);
Translation of the reflecting element operating a single translation motion along a measuring direction in such a way as to obtain the generation of two interference peaks (30, 40) in correspondence of the reaching of the two interference positions in which respectively the optical distance of the first surface (8) from the source (5) is equal to that of the external surface of the eye (100) with the source (4) and the optical distance of the second surface (9) from the source (5) is equal to that of the source (4) from the retina;
And wherein the said two surfaces (8, 9) are arranged in the arrangement (7) in such a way as to keep said reciprocal distance (d) constant at least during the translation of the arrangement (7) in the scanning phase and so that the first surface (8) generates the first interference peak at the beginning of the translation and afterwards during said translation the second plate (9) generates the second interference peak.

Advantageously, an operation of acquisition of a plurality of images at different focal distances is preliminarily foreseen, with a consequent translation of the interferometer on the basis of the optimal focal distance obtained so that when the image acquired results in focus the interferometer results positioned at a distance in which the first surface (8) substantially generates interference immediately with respect to the first surface (100) of the eye.

Advantageously, the distance between the first and the second surface (8, 9) is fixed and is not modified when the measurings to make of the intraocular distances vary.

Advantageously, the distance between the first (8) and the second surface (9) is comprised between the 12 mm and the 19 mm.

### BRIEF DESCRIPTION OF DRAWINGS

Further features and advantages of the present device and relative method, in accordance with the invention, will result clearer with the description that follows of some preferred embodiments, made to illustrate but not to limit, with reference to the annexed drawings, wherein:

FIG. 1 and FIG. 2 show a schematization of the Michelson interferometer in accordance with the present invention;

FIG. 3 schematizes the detecting phase of the first peak;

FIG. 4 schematizes the detecting phase of the second peak;

FIG. 5 shows schematically the overall graph with the relative peaks (double sheet) and, just as a way of example, shows how the graph is a sort of overlapping between two interferometers that work independently and simultaneously.

FIG. 6 schematizes graphically the calculation that allows to obtain the axial distance sought;

FIG. 7 shows a possible technical solution for the movement of the arrangement 7 provided with the two plates (8, 9);

FIG. 8 shows the frame on which the interferometer is arranged and its translation in order to obtain an image of the eye that results in focus.

### DESCRIPTION OF SOME PREFERRED EMBODIMENTS

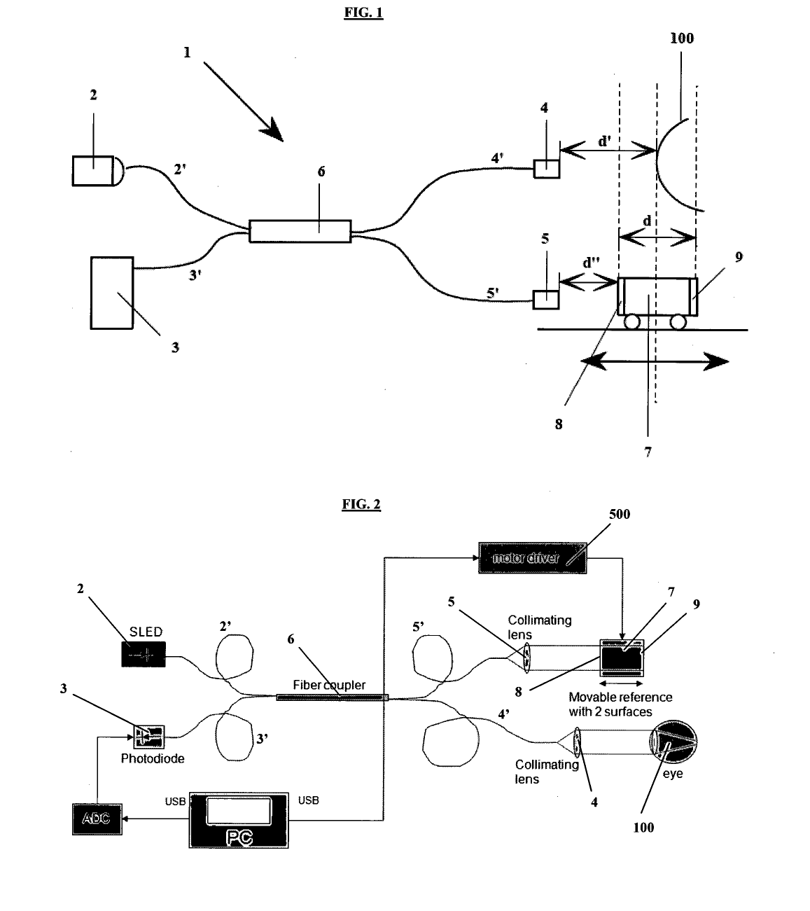

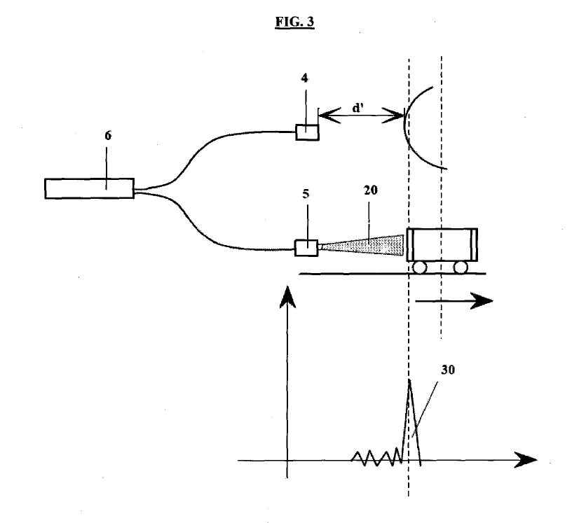

With reference to FIG. 1 and to FIG. 2, an interferometer 1 is described in accordance with the present invention.

The interferometer, as per the background art, foresees a coupler 6 (“fiber coupler” as per FIG. 2) to which the four branches of optical fiber converge (2′, 3′, 4′, 5′). A first branch 2′ is connected to a source 2 (SLED in FIG. 2) and a second branch 3′ is connected to the receiving photodiode 3 (Photodiode 3 in FIG. 2) that elaborates in return the reflected light beam. On the opposite part to the node 6 the other two branches of optical fiber (4′, 5′) converge and of which one (the branch 4′) connected to the collimator 4 placed opposite the eye 100 and the other one (the branch 5′) connected to a collimator 5 placed opposite a reflecting element 7.

FIG. 2, as already described in the preamble of the background art, also describes the electronic processor (PC) which is placed in communication with the photodiode 3 through a converter (ADC).

As per the state of the art, therefore, the beam emitted by the source 2 is sent in part to the eye 100 and in part to the reflecting element 7 through the two ramifications (4′, 5′) in such a way that the photodiode 3, in return, receives the beam reflected through the branch 3′ and analyzes the interference between said reflected waves.

In accordance with the invention, the reflecting element 7 (also called reflecting arrangement 7) foresees a first plate 8 and a second plate 9 at least partially reflecting between them, parallel and distanced of a pre-determined fixed quantity (d). The plates are preferably flat.

In the preferred configuration of the invention the reflecting element 7 has the shape of a cylinder whose anterior face foresees the first reflecting plate 8, while the posterior face constitutes the second reflecting plate, which are coaxial between them (apart from being, as already said, parallel).

The cylinder 7, as schematically shown in FIG. 1 and in FIG. 2, is therefore translatable along a sliding binary and in accordance with the double direction of the arrow always shown in FIG. 1 and in FIG. 2. In this way, the translation of the cylinder causes an integral translation of the two reflecting faces (8, 9) which, belonging to the cylinder and being fixed to it, keep their reciprocal distance (d) unvaried during all the translation.

Always FIG. 2 also shows a guide engine 500 (motor driver of FIG. 2) which is activated by the PC and controls the movement of the cylinder.

The cylinder 7, for the purposes of the following invention, can have lengths preferably comprised within a range between 12 mm and 19 mm and, preferably, a length of about 12.5 mm in such a way as to ensure the maximum decoupling between the positions where the maximums measured of cornea and retina are, in normal conditions, diminishing.

It is reminded that, actually, the distances mentioned here are the geometricals of the cylinder and therefore, as the preferred embodiment of the invention foresees the use of a standard glass (BK7, with a refraction index n=1.5), the optical distances are those geometrical multiplied by said factor n of 1.5. Basically, the optical length is given by the product of the geometrical length with refraction index of the material measured at 820 nm. With the values of the geometrical length of the cylinder within said ranges, a value of optical length of the optimal cylinder comprised between 15 mm and 21 mm can be extrapolated.

Obviously, other sizes of cylinder and other types of glass could be used without for this moving apart from the present inventive concept.

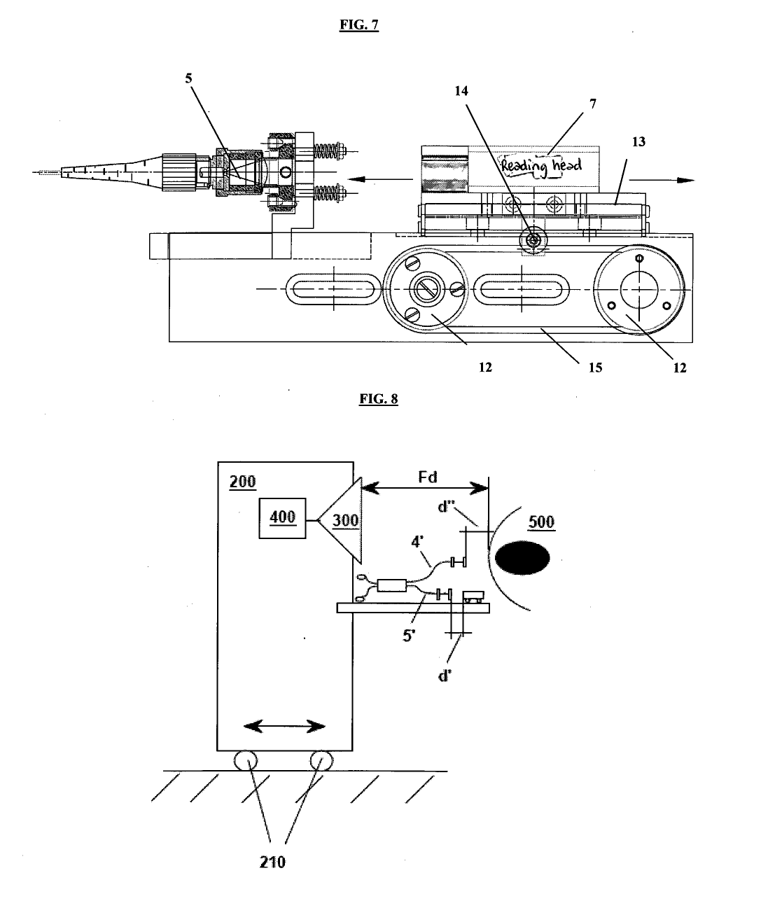

FIG. 7 shows structurally a constructive solution adopted to allow the translation of the arrangement 7. The solution shows two wheels 12 that are rotatable around their hinge axis and connected by a dragging element, for example a belt 15. A motorized system, for example electric, is used for commanding the rotation of one or both wheels 12, therefore causing the dragging in motion of the belt 15. A grasping element 14, for example a pair of opposed nut, pliers or a pivot that is inserted in the belt, is integral to the slide 13 and is interposed between the slide 13 and the belt 15. The element 14 grasps the belt in such a way as to allow that the belt drags the slide during its alternate motion. As shown in FIG. 7, the two directions of alternate motion from the arrangement 7 are highlighted.

In a possible variant of the invention, it would be possible to foresee that the arrangement 7, for example in the shape of a cylinder of other shape, foresees a system of adjustment of the reciprocal distance (d) between the two plates (8, 9). For example, a simple sliding system, assembling the two plates on appropriate binaries within the cylinder 7, would allow to adjust their reciprocal distance (d). A blocking system then allows to fix the selected position so as to conduct the scanning with said fixed distance (d).

This solution, although structurally more complex, allows to adapt the arrangement 7 to particular biometric characteristics of the patient under examination, for example in the case of children in which the distances to measure could be different from those of an adult.

Although in all the embodiments described it has been indicated that, preferably, the two plates are coaxial between them, actually nothing would impede to arrange them in a non-coaxial way. It is in fact enough that the beam coming in in the first plate intercepts also the second plate without their being obligatorily perfectly coaxial.

A further variant could foresee that the two plates are arranged in such a way as not to result either coaxial or parallel between them. This solution would be possible arranging, for example, the second plate at a right angle with respect to the first one, therefore forming a corner. In this case, it would be enough to arrange a reflecting element in such a way that the last one reflects the entering beam through the first plate on the second one placed at a right angle with respect to the first one.

Moreover, it is clear that equivalent solutions can anyway foresee different shapes of the reflecting element, for example not a cylinder but a parallelogram.

In use, therefore, the functioning is the following.

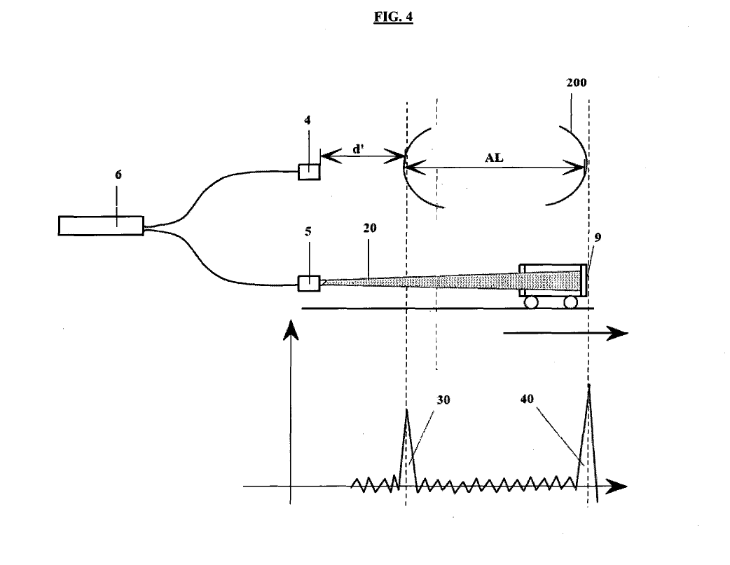

Initially, FIG. 1 shows an initial position in which the cylinder 7 is placed at such a distance from the collimator 5 that the first plate 8 results at a distance (d″) inferior to the distance (d′) between the collimator and the surface 100 of the eye. In this way, on the basis of the appropriate geometrical length of the cylinder, it is as if virtually the cylinder 7 resulted between the external surface of the eye, that is the surface of the vitreous humour 100. By the term “between” it is intended that the first plate is placed at an optical distance inferior with respect to the surface of the cornea, while the second plate is at an optical distance superior to the cornea but inferior to the retina. The first reflecting surface 8 will therefore be found in a backward position with respect to the eye 100 exactly as in the initial position of the device discussed in the state of the art. At this point, the translation initiates up to when the first reflecting surface 8 reaches the first equilibrium distance (equivalent optical paths) generating, through a ray reflected by the beam 20, the first interference peak 30 (see FIG. 3).

As discussed in the state of the art, the temporal tracing of the signal obtained by the interferometer during the translation of the reference plates contains a series of maximums, in correspondence of all the axial positions of the intraocular surfaces to measure. In particular, there is the first intense maximum precisely in correspondence of reaching the first plate 8 in correspondence of the anterior surface of the cornea. All the maximums are surrounded by an envelope having an oscillating shape and caused by secondary minimums and maximums of the interference, due to the partial coherence of the light emitted by the SLED. Known algorithms of envelope therefore allow the extraction of the maximum.

The plates are made of untreated glass, reflecting in a range variable between 1% and about 4%, preferably 4%. In this way, the plates are not darkening and the light passes and is reflected also by the posterior plate 9. The system would not work with mirrors or high-reflectance plates since in that case the posterior plate would not be hit by any beam, which is instead totally screened by the anterior plate.

Going on with the translation the cylinder reaches the surface of the retina 200 (see FIG. 4). In particular, the second plate 9, when it reaches an optical path equivalent to that of the retina, it reflects the beam 20, generating the second interference peak 40 (see FIG. 4). The 4% of energy that returns from each plate is more than enough to make the measuring. Obviously, the second surface 9 is hit by the 96% of the radiation that affects the first one 8 (if the first plate is reflecting at 4%).

Thanks to the fact that the cylinder has a pre-determined geometrical length L the second reflecting surface will intercept the retina (that is the distance of equilibrium in which the optical paths are equal) much before with respect to the background art, that is the two peaks will be much nearer between them.

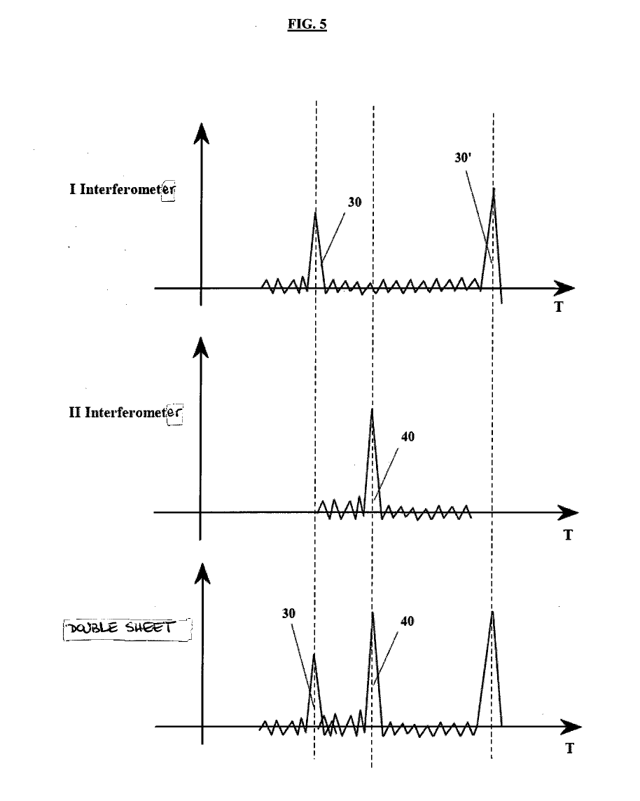

For that purpose, FIG. 5 clarifies this since it assimilates said system with two interferometers that work independently. It is therefore highlighted that the first plate 8, once the cornea is intercepted, generates the peak 30. The peak 30′ is that one that is generated when the same plate reaches the optical distance corresponding to the position of a second reflecting ocular surface, for example the retina. In this case, as per the background art, the time T required for the measuring would be long.

The second interferometer, however, has its own plate 9 placed at an optical path near the retina. Therefore, it generates almost immediately after the generation of the first peak 30, the second peak 40. The double sheet or plate, in accordance with the invention, therefore generates a signal that foresees the two peaks 30 and 40 very near between them and therefore obtained in a halved T time with respect to a system of the background art.

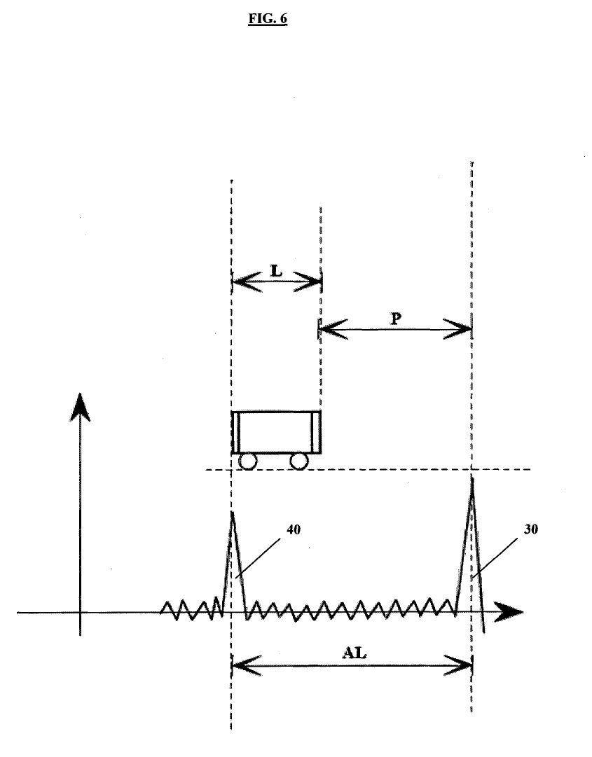

In this way, as schematically shown in FIG. 6, the distance AL sought is now easily implemented with a calculation that takes advantage of the distance P covered by the cylinder, distance in which the cylinder 7 has generated the two peaks (30, 40), summed to the length L of the cylinder (this multiplied appropriately by the index of refraction of the cylinder glass) and the whole divided by the index of refraction of the eye.

It is not necessary, therefore, as described in the background art, to make a high-frequency oscillation, or every time to adjust the distances between the two plates to that that is the distance that is expected. Starting from an initial position a single stroke in one direction is enough to have a tracing that allows to obtain to the desired measure.

It is then eventually possible to repeat the single motion with a second or more strokes (for example three) which eventually serve, but not necessarily, to obtain a verification tracing. It is obvious that, nevertheless, it is not a high-frequency oscillation in which innumerable strokes per minute are made, forward and return ones, at a precise temporal cadence.

As per FIG. 1, the PC controls the movement of the cylinder by means of a guide engine 500. In particular, the PC memorizes initial and final positions of movement, always commanding such a translation between these two extreme points each time an optical measuring is commanded.

An important aspect of the present invention, as schematically shown in FIG. 8, concerns the initial positioning of the Michelson interferometer with respect to the eye 500. To that aim, the interferometer 1 (schematized in figure) is assembled on a structure 200 that foresees a manual or automatic translation system 210 (double direction of the arrow). On the structure 200 the entire interferometer 1 and an apparatus of acquisition of the image (300, 400) are placed. There exist and have long been known for ophthalmological equipment apparatus for the acquisition of images since they are used to reconstruct shapes of optical surfaces. In this case, the acquisition of the image is used in an innovative way.

The acquisition of the image can be made in various different ways. For example, in a case, through the arrangement of luminous LED directly on the Placido disk 300. The LED thus project the light on the ocular cornea.

Alternatively, a luminous “pattern” can be used obtained with appropriate LED positioned in different spots of the Placido disk and that also project a luminous “pattern” on the surface of which the image wants to be acquired.

A videocamera 400 acquires the images that are analyzed through an appropriate software.

The translation system 210 is motorized and is controlled manually or automatically in such a way as to cause ad advancement and a retrocession of the entire structure 200 on which the interferometer 1 in arranged with respect to the surface 500 to acquire, thus acquiring various images (for example 25 frames per second) at different focal distances (Fd). Such images are analyzed with known software techniques to find the optimal focus distance (Fdc). By optimal focus distance it is intended the distance at which the videocamera takes an image perfectly in focus. Analysis algorithms to evaluate the quality of focus of an image are already known and therefore will not be further described in detail here.

Therefore, in a manual way the user can move in a direction or in the opposite direction the structure 200 until the position in which the software indicates that the optimal focus position has been reached, or, alternatively, this can take place automatically.

In accordance with the invention, therefore, the interferometer 1 is arranged on the structure and therefore translates integrally to the structure 200 during the search of the image in focus. In particular, the interferometer is placed in such a way so that when the image results in focus it is at a distance in which the optical path of the light reflected by the cornea 100 along the branch 4′ is equal to the optical path of the light reflected from the first plate 8 in the branch 5′ (equal optical distances d′ and d″). Basically, when the image is in focus the distance of the interferometer from the cornea is substantially such as to generate immediately the first interference peak.

It is reminded here that the frame 200 foresees a support base for the chin of the patient, so that the ocular distance from the videocamera and from the interferometer can easily be adjusted as said above.

This adjustment of initial position of the interferometer, which takes advantage of an acquisition of an image in focus, has the advantage of speeding up the process of measuring of the distance, rendering it precise above all.

First of all, any ambiguity is eliminated with respect to the background art since the first peak that is detected is certainly that of cornea 100. All the other peaks are surfaces internal to the eye with respect to which the distance wants to be measured. The translation stroke becomes in this way very efficient since a single stroke allows to measure also many distances of different surfaces and it is not necessary anymore each time to have to adjust the two plates at a distance that is the one that is expected to be measured. It is enough to adjust or select two plates at a single pre-chosen distance of a value inferior to the measurings to make and the same plates, with a single stroke, can be used from the starting point to scan all the surfaces of interest with a single stroke.

Basically, being the interferometer positioned in a known initial condition in which the first interference peak is quickly found, then the entire stroke can be taken advantage of to find other ocular surfaces, thus increasing the range of intraocular axial measuring.

In the present description the term luminous beam indicates in a totally generic manner and not limiting a beam that, as said, is generally in the field of the infrared.

1. A Michelson-type interferometer (1) for measuring the intraocular axial length (AL) and comprising:
An arrangement (7) at least partially reflecting of the light, translatable along a motion direction (P) so as to be able to move apart and come near with respect to an emitting light source (5), said arrangement (7) comprising at least a first surface (8) and at least a second surface (9) at least partially reflecting and arranged between them at a pre-determined reciprocal distance (d);
Said two surfaces (8, 9) being arranged in the arrangement (7) so as to keep constant said reciprocal distance (d) at least during the translation of the arrangement (7) in the scanning phase;
A motorized driving system (500) to command the movement of the reflecting arrangement (7);
Characterized in that said motorized driving system (500) is controlled in such a way that the scanning is completed in a single translation motion in a direction starting from an initial position in which the first surface (8) generates the first interference peak at the beginning of the translation and with such a fixed distance (d) between the plates that afterwards during said translation the second plate (9) generates the second interference peak.

2. An interferometer (1), as per claim 1, wherein the first (8) and the second surface (9) are placed parallel between them.

3. An interferometer (1), as per claim 1 or 2, wherein the first (8) and the second surface (9) are placed coaxial between them.

4. An interferometer (1), as per one or more of the preceding claims, wherein adjustment means are foreseen for allowing a reciprocal sliding of the two surfaces (8, 9) in the arrangement (7) in such a way as to adjust their reciprocal distance (d) and fixing means for blocking said reciprocal distance.

5. An interferometer (1), as per one or more of the preceding claims from 1 to 3, wherein the two surfaces (8, 9) are fixed.

6. An interferometer, as per one or more of the preceding claims, wherein said reflecting arrangement (7) is cylindrical.

7. An interferometer, as per one or more of the preceding claims, wherein said reflecting arrangement (7) has an optical length comprised between 15 mm and 25 mm.

8. An interferometer, as per one or more of the preceding claims, wherein the distance between the first (8) and the second surface (9) is comprised between 12 mm and 19 mm.

9. An interferometer, as per claim 1, wherein the motorized system (500) is connected to an electronic processor (PC) that commands the motion within two extreme positions.

10. An interferometer (1), as per one or more of the preceding claims, wherein the two reflecting surfaces (8, 9) are of low reflectance in such a way as to reflect in a range comprised between the 1% and the 4% of the beam that hits them, and preferably of the 4%.

11. An interferometer (1), as per one or more of the preceding claims, wherein the following are further foreseen:
A system of acquisition/analysis of the images (300, 400) configured to acquire a plurality of images at different focal distances and select the image that results in focus;
A translation system (210) that allows to translate the interferometer (1) on the basis of the images acquired in such a way that when the image acquired results in focus the interferometer results positioned with the first surface (8) substantially at an interference distance with respect to the first surface (100) of the axial length (AL) to be detected.

12. An interferometer (1), as per claim 11, wherein a frame is foreseen on which said acquisition/analysis system of the images and the interferometer (1) are arranged, said frame being reciprocable in such a way as to allow the acquisition of images at different focal distances.

13. A method for measuring the intraocular axial length (AL) by means of a Michelson-type interferometer (1) comprising an arrangement (7) at least partially reflecting the light and provided with at least a first (8) and at least a second (9) surface at least partially reflecting, arranged between them at a pre-determined reciprocal distance (d), said method comprising the following operations:
Initial positioning of the reflecting arrangement (7) in such a way that the first reflecting surface (8) is found at a distance (d″) from the collimator (5) inferior to the distance (d′) of collimator (4) from the cornea (100) of the eye;
Sending of the light beam through the two branches (4′, 5′) terminating in the two collimators (4, 5);
Translation of the reflecting element operating a single translation motion along a measurement direction in such a way as to obtain the generation of two interference peaks (30, 40) in correspondence of the reaching of the two interference positions in which respectively the optical distance of the first surface (8) from the source (5) is equal to that of the external surface of the eye (100) with the source (4) and the optical distance of the second surface (9) from the source (5) is equal to that of the source (4) from the retina;
And wherein the said two surfaces (8, 9) are arranged in the arrangement (7) in such a way as to keep constant said reciprocal distance (d) at least during the translation of the arrangement (7) in the scanning phase and so that the first surface (8) generates the first interference peak at the beginning of the translation and afterwards during said translation the second plate (9) generates the second interference peak.

14. A method, as per claim 13, wherein an operation of acquisition of plurality of images at different focal distances is preliminarily foreseen with a consequent translation of the interferometer on the basis of the optimal focal distance obtained so that when the image acquired results in focus the interferometer results positioned at a distance in which the first surface (8) substantially generates right after interference with respect to the first surface (100) of the eye.

15. A method, as per claim 13 or 14, wherein the distance between the first and the second surface (8, 9) is fixed and is not modified when the measurements of the intraocular distances to make vary.

16. A method, as per one or more of claims from 13 to 15, wherein the distance between the first (8) and the second surface (9) is comprised between 12 mm and 19 mm.
